(19) United States
(12) Patent Application Publication
(10) Pub. No.: US 2013/0030286 A1
(43) Pub. Date: Jan. 31, 2013

(54) IMAGE GUIDED SURGERY TRACKERS USING MULTIPLE ASYNCHRONOUS SENSORS

(71) Applicants: Ali T. Alouani, Cookeville, TN (US); Brian Lennon, Nashville, TN (US); Ben Neese, Nashville, TN (US); James Stefansic, Nashville, TN (US)

(72) Inventors: Ali T. Alouani, Cookeville, TN (US); Brian Lennon, Nashville, TN (US); Ben Neese, Nashville, TN (US); James Stefansic, Nashville, TN (US)

(21) Appl. No.: 13/555,144

(22) Filed: Jul. 21, 2012

Related U.S. Application Data
(60) Provisional application No. 61/512,484, filed on Jul. 28, 2011.

(51) Int. Cl. A61B 6/12

(52) U.S. Cl. 600/424

(57) ABSTRACT

An apparatus and related methods using a variety of heterogeneous sensors to accurately track, in real time, the location of the tip of a surgical instrument inside the human body. The system accounts for real time changes in the surrounding environment during surgery, and when integrated with noninvasive image-guided surgery (IGS), this invention makes IGS possible and safe without tedious offline calibration. Sensors include, but are not limited to, optical, electromagnetic (EM), and sonar.

This application claims benefit of and priority to U.S. Provisional Application No. 61/512,484, filed Jul. 28, 2011, by Ali T. Alouani, et al., and is entitled to that filing date for priority. The specification, figures and complete disclosure of U.S. Provisional Application No. 61/512,484 are incorporated herein by specific reference for all purposes.

### FIELD OF INVENTION

This invention relates to an apparatus and a method that uses a variety of heterogeneous sensors to accurately track, in real time, the location of the tip of a surgical instrument inside the human body. It accounts for real time changes in the surrounding environment during surgery, and when integrated with noninvasive image-guided surgery (IGS), this invention makes IGS possible and safe without tedious offline calibration.

### BACKGROUND OF THE INVENTION

In current medical practice, surgeons often use an open cavity to perform a surgery. This invasive procedure, besides being unnecessarily costly, impacts recovery time, the risk of infections, and the psychology of the patient. To overcome some of the limitations of open cavity surgeries, the concept of minimally invasive surgery (MIS) has been pursued, as disclosed, for example, in U.S. Pat. No. 5,381,782 (incorporated herein by specific reference in its entirety for all purposes). The benefits of minimally invasive surgery include reduced surgical procedure pain, patient anxiety, and post-operative recovery time.

A prerequisite for the success of image-guided surgery (IGS) systems is the correct display of the position of a surgical instrument on a preoperatively or intraoperatively acquired image of the patient. This is accomplished by accurately tracking an instrument and mapping or registering it to the patient image space. There are various methods by which to do this. One method commonly used in IGS is optical tracking Using a three dimensional spatial localizer, the position and orientation of the tip of a surgery probe can be obtained with an accuracy of less than approximately 1.0 mm whenever four or more infrared-emitting diodes (IREDs) are visible. Such tracking accuracy is very good for surgical applications with rigid instruments. Unfortunately, often times during surgery the line of sight can be lost and optical tracking techniques can be inaccurate, especially when flexible instruments are used.

In order to track flexible probes inside the human body, electromagnetic tracking has been introduced. Such trackers are not dependent on a free line-of-sight. However, due to magnetic field distortions resulting from the presence of magnetic fields generated by eddy currents in conductive objects and electronic equipment that exist in any surgery room, the accuracy of a magnetic tracker can be unsatisfactory. Several techniques have been proposed to correct for the errors in electromagnetic trackers. These correction methods attempt to estimate the distortion over the work space volume. Besides being tedious and time consuming, they assume that the distortion is fixed over a long period of time.

To reduce the error of magnetic trackers, the idea of using hybrid trackers has been introduced. The basic idea is to simultaneously measure the position and orientation of specific locations in the line-of-sight, using both the optical tracker (OT) and the electromagnetic tracker (EMT). Then the difference between the sensors measurements, in a common reference frame, is used to calibrate the EMT.

Existing techniques of magneto-optic trackers use the optical tracker measurement as a reference to model the magnetic distortions. For this purpose, several measurements are carried out across the distorted region to model the magnetic distortion using polynomials with different degrees. Thousands of measurements are needed to perform calibration before the medical procedure starts. This tedious process has to be repeated for different surgery rooms and even for the same room every time equipment is moved. Furthermore, the calibration is done offline and does not account for the error of the distortion model.

In target tracking applications, one does not have the luxury of modeling the disturbances in the air space offline. Accordingly, what is needed is real time target tracking without a priori knowledge of the target model and disturbances to which the moving target is subject to.

### SUMMARY OF INVENTION

In various embodiments, the methods of the present invention treat the tip of a minimally invasive surgical instrument as a moving target inside the body, and is tracked in real time using an array of heterogeneous sensors, such as, but not limited to, optical, electromagnetic (EM), and sonar. The tracking of the minimally invasive instrument tip is accomplished without a priori knowledge about the target trajectory and target dynamics.

To increase the accuracy of the tracking system, more than one sensor may be used. Long range sensors, for example, can be used to detect the presence of a potential target in a region or space, but may not provide accurate measurements of the position of the target. Short range sensors can provide that accurate position information, but are not able to detect the presence of the target while it is far away. The use of both long range and short range sensors can lead to the design of a successful system that is not possible when only one of the sensors is used alone.

In image-guided surgery, optical sensors provide accurate position information about the instrument tip in open surgery. However, such sensors cannot accurately track a flexible instrument whenever it is inside the human body in either an open or minimally invasive fashion. On the other hand, sensors such as electromagnetic (EM) sensors can provide position information in the absence of line-of-sight. However, such sensors are sensitive to magnetic distortions. When used alone, each type of sensor can exhibit an accuracy degradation. When used together, accurate tracking becomes possible even in the absence of line of sight and in the presence of magnetic distortions.

In another embodiment, a real-time imaging modality, such as ultrasound or any other sensing mechanism, may also be incorporated into the system. By tracking the imaging device, the real-time image can be located in physical space by utilizing an image-to-space calibration. By defining the locations of important features (e.g., tool tip, tool shaft) in the image, the same features can be localized in physical space. The coordinates of these features can then be presented as additional inputs to the filter. This information serves to further correct the tracking error and more accurately define the location of the tool tip.

### BRIEF DESCRIPTION OF THE DRAWINGS

FIG. 1 shows a view of the architecture of a real time tracking system of a minimally invasive instrument tip using an array of heterogeneous sensors in accordance with an embodiment of the present invention.

FIG. 2 shows a view of a minimally invasive instrument with embedded EM sensors in accordance with an embodiment of the present invention.

FIG. 3 is a diagram of steps for a real time calibration and tracking method using an optical and three EM sensors in accordance with an embodiment of the present invention.

FIG. 4 is a diagram of online calibration method in accordance with an embodiment of the present invention.

### DETAILED DESCRIPTION OF EXEMPLARY EMBODIMENTS

In various exemplary embodiments, the methods of the present invention treat the tip of a minimally invasive surgical instrument as a moving target inside the body, and is tracked in real time using an array of heterogeneous sensors, such as, but not limited to, optical, electromagnetic (EM), and sonar. The tracking of the minimally invasive instrument tip is accomplished without a priori knowledge about the target trajectory and target dynamics.

To increase the accuracy of the tracking system, more than one sensor may be used. Long range sensors, for example, can be used to detect the presence of a potential target in a region or space, but may not provide accurate measurements of the position of the target. Short range sensors can provide that accurate position information, but are not able to detect the presence of the target while it is far away. The use of both long range and short range sensors can lead to the design of a successful system that is not possible when only one of the sensors is used alone.

In image-guided surgery, optical sensors provide accurate position information about the instrument tip in open surgery. However, such sensors cannot accurately track a flexible instrument whenever it is inside the human body in either an open or minimally invasive fashion. On the other hand, sensors such as electromagnetic (EM) sensors can provide position information in the absence of line-of-sight. However, such sensors are sensitive to magnetic distortions. When used alone, each type of sensor can exhibit an accuracy degradation. When used together, accurate tracking becomes possible even in the absence of line of sight and in the presence of magnetic distortions.

The challenges in such a combination are due to the fact that, in general, heterogeneous sensors, such as the aforementioned electromagnetic and optical sensors, have different data rates and use independent clocks to generate the measurements. For this reason they are called asynchronous sensors. Furthermore, communication delays exists in the data generation, collection and processing of such sensors. These challenges have prevented prior art systems from effectively using such sensors together in the same tracking system.

In another embodiment, a real-time imaging modality, such as ultrasound or any other sensing mechanism, may also be incorporated into the system. By tracking the imaging device, the real-time image can be located in physical space by utilizing an image-to-space calibration. By defining the locations of important features (e.g., tool tip, tool shaft) in the image, the same features can be localized in physical space. The coordinates of these features can then be presented as additional inputs to the filter. This information serves to further correct the tracking error and more accurately define the location of the tool tip.

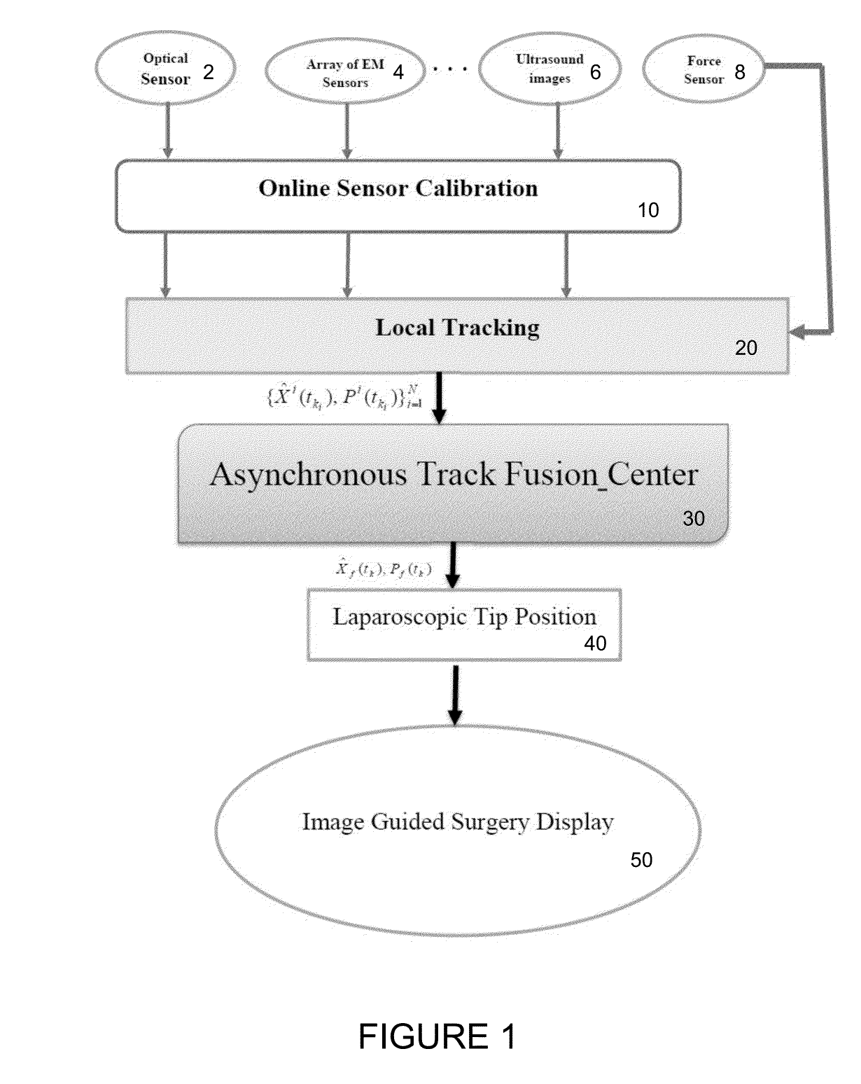

An example of a general tracking system is depicted in FIG. 1. Sensors and input mechanisms include optical sensors 2, EM sensors 4, ultrasound 6, and force sensors 8. The sensors are calibrated online 10, and local tracking data is incorporated 20. The asynchronous track fusion center 30 processes the data to determine the position of the instrument tip (in this example, a laparoscopic instrument, although any other minimally invasive instrument may be used) 40, and displays it on the IGS display 50.

The moving minimally invasive instrument tip can be either in linear motion or maneuvering mode. In one exemplary embodiment as a medical application, the linear motion assumes a constant velocity motion, while the maneuvering mode takes place whenever the instrument is deflected. In a Cartesian coordinates system, the tip dynamics can be modeled as

\dot{X}(t)=AX(t)+GW(t) (1)

where X represents the state (position and orientation) of the minimally invasive tip, and W is a random process that models uncertainties about the tip dynamics. Typically, W is assumed to be independent Gaussian with zero mean and covariance Q(tₖ). Assume that the minimally invasive tip position is observed by a number of sensors, such as optical, electromagnetic, sonar, and the like. These sensors have different data rates and a different clock system. Let

zⁱ(tₖ_{ᵢ})=hⁱ(X(tₖ_{ᵢ})+Vⁱ(tₖ_{ᵢ}), i=1, 2, . . . N (2)

be the measurement taken by sensor #i at time tₖ_{ᵢ}. Vⁱ(tₖ) is the measurement noise of sensor #i that is assumed to be white Gaussian with covariance Rⁱ(tₖ_{ᵢ}). This covariance can be determined using the accuracy information provided by the sensor manufacturer. Note that the different sensors measurements may be taken at different time since these sensors may have different data rates and use different clocks. To track the minimally invasive instrument tip position by each sensor, an extended Kalman filter or unscented Kalman Filter (such as discussed in Jazwinski, A. H. Stochastic Processes and Filtering Theory. New York, Academic Press, 1970; Y. Bar-Shalom and R. Li, Estimation and Tracking, Artech House, 1993; S. J. Julier and J. K. Uhlmann, “Unscented Filtering and Nonlinear Estimation,” Proc. IEEE, vol. 92, no. 3, 2004; and T. Lefebvre, H. Bruyninckx, and J. de Schutter, “Kalman Filters for Non-Linear Systems: A Comparison of Performance,” Int'l J. Control, vol. 77, no. 7, pp. 639-653, 2004; all of which are incorporated herein by specific reference in their entireties for all purposes) can be used to estimate the tip position and velocity, called here the local track, using the dynamical and measurement models given by Eq. (1) and the corresponding sensor measurement model presented in Eq. (2), respectively. During a given period of time, each local tracker may produce a single or multiple local tracks. This is due to the difference in the data rate of the different sensors. These local tracks may be produced at different times due to the asynchronicity of the sensors and the communication delays between the sensors and their corresponding local processors.

Let X(t) be the true state of the minimally invasive instrument tip (position, orientation, and velocity) at time t. Let \hat{X}ⁱ(tₖ_{ᵢ}) be the estimate of the state of the tip as provided by the local tracker #i that uses the measurements from sensor #i , zⁱ(tₖ_{ᵢ}), and let \tilde{X}ⁱ(tₖ_{ᵢ}) be the error in the state estimate of tracker #i:

\tilde{X}ⁱ(tₖ_{ᵢ})=X(tₖ_{ᵢ})−\hat{X}ⁱ(tₖ_{ᵢ}), tₖ₋₁≦tₖ_{ᵢ}<tₖ, i=1, 2, . . . , N (3)

The error covariance matrix of the tip state produced by local tracker #i is defined as

Pⁱ(t)=E[\tilde{X}ⁱ(t)\tilde{X}ⁱ(t)^{T}] (4)

The error covariance is a measure of the error in the estimate of the tip state as produced by local tracker #i.

Given a number of local tracks of the minimally invasive instrument tip at different times, the objective is to find the best track in the minimum mean square sense by fusing all the incoming local tracks. The solution to this problem is an adaptation of the solution of a general distributed state estimation problem using multiple asynchronous sensors with communication delays, as disclosed in Alouani, A. T. and J. E. Gray, “Theory of distributed estimation using multiple asynchronous sensors, IEEE Transactions on Aerospace and Electronic Systems, Vol. 41, No. 2, April 2005 (a copy of which is appended hereto as incorporated herein by specific reference in its entirety for all purposes). This solution was applied to target tracking in military applications, as disclosed in A. Alouani, et al., U.S. Pat. No. 7,884,754, which is incorporated herein by specific reference in its entirety for all purposes.

The solution to this problem is summarized as follows. Given the asynchronous local tracks, (\hat{X}ⁱ(tₖ_{ᵢ}), Pⁱ(tₖ_{ᵢ}))ᵢ₌₁^{N}, tₖ₋₁≦tₖ_{ᵢ}≦tₖ, i=1, 2, . . . , N of the minimally invasive instrument tip, the optimal track of the tip state, (\hat{X}_{f}(tₖ), P_{f}(tₖ)), in the minimum mean square sense at time tₖ is given by:

$${\hat{X}}_{f} ( {t}_{k} ) = {\sum }_{i = 1}^{N} {L}_{i} {\hat{X}}^{i} ( {t}_{{k}_{i}} ) \\ {P}_{f} ( {t}_{k} ) = {\sum }_{i = 1}^{N - 1} {\sum }_{j = 1}^{N - 1} {L}_{i} {M}_{ij} {L}_{j}^{′} + {\sum }_{i = 1}^{N - 1} {L}_{i} {N}_{i} + {\sum }_{i = 1}^{N - 1} {N}_{i}^{′} {L}_{i}^{′} + {M}_{n} \qquad ( 5 )$$

where (Lᵢ)ᵢ₌₁^{N} are weighting matrices used to assign different weights to the different local tracks to achieve the best fused track.

It is important to note that due to the sensors' asynchronicity, the local tracks, (\hat{X}ⁱ(tₖ_{ᵢ}), Pⁱ(tₖ_{ᵢ}))ᵢ₌₁^{N}, tₖ₋₁≦tₖ_{ᵢ}≦tₖ, i=1, 2, . . . , N are generated at different times; the times when the local measurements were taken. Furthermore, the local tracks may arrive at the track fusion center at times different from the times they were generated as a result of communication delays. The track fusion algorithm provided in Eq. (5) is optimal in the presence of sensor asynchronicity. In addition, the communication delays do not affect the optimality of the fused track as long as the local tracks arrive on or before the fusion time tₖ. Further details may be found in the Alouani reference incorporated above.

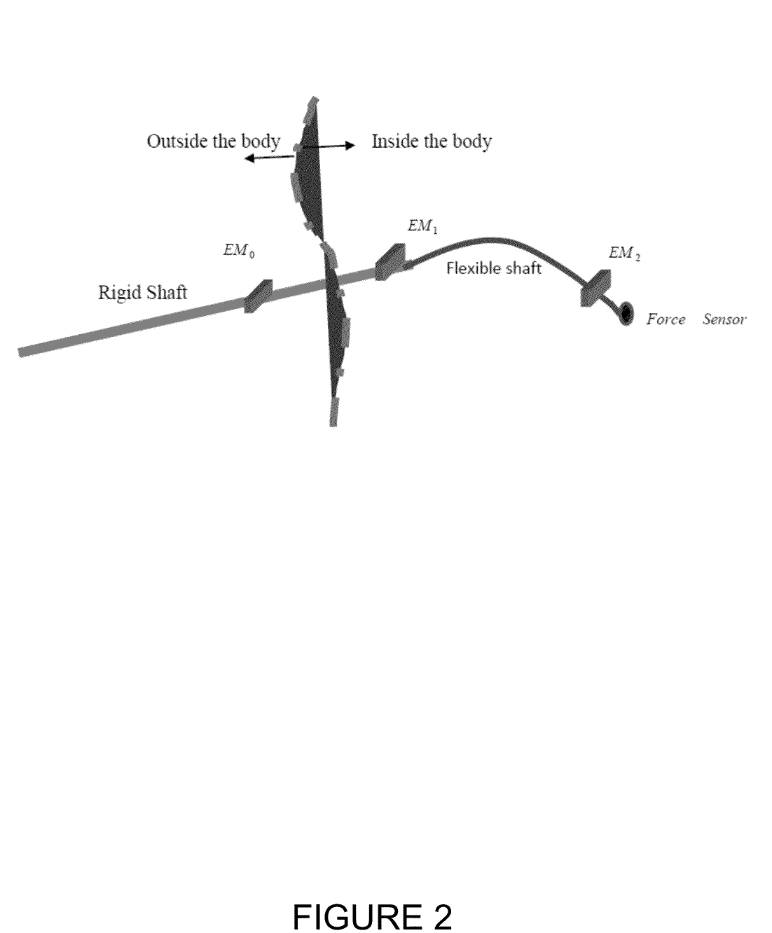

In one exemplary embodiment, a minimally invasive tool or instrument is made up of solid and flexible sections, as seen in FIG. 2. It is equipped with three or more electromagnetic sensors. Sensors EM₀ and EM₁ are located on the solid section of the instrument. EM₀ remains in the line-of-sight of the optical tracker (OT) at all times. EM₁ is located at the end of the solid section and may or may not be in the line-of-sight of the optical tracker during surgery. EM₂ is located at the tip of the instrument. Other sensors, such as a pressure sensor, may be added to further improve the tracking accuracy of the minimally invasive tip position, especially in detecting the start of a deflection.

Since the sensor EM₀ is always in the line of sight of the optical tracker, it can be continuously tracked optically without impact from magnetic distortion. Given that EM₁ is on the rigid shaft of the minimally invasive instrument, its position can be determined by simple transformation of the position of EM₀. Similarly, before deflection of the tip, the position of EM₂ can be computed using the optical measurement of EM₀. Therefore, the position of EM₀ and EM₁ can be provided by the optical tracker during the whole surgery. In the presence of magnetic distortion, the measurements provided by EM₀ and EM₁ will be different from the ones provided by the optical tracker. The difference between these measurements will be used to estimate the magnetic distortion in real time.

The online calibration algorithm uses the asynchronous data provided by the optical and electromagnetic sensors to estimate the magnetic distortion, called here bias, as the minimally invasive instrument moves inside the body. Assuming that data rate of the EM tracker is higher than that of the optical tracker, between two consecutive measurements of the optical sensor, each EM sensor takes a number n of measurements of its position. In what follows, the online calibration of EM is considered. The same approach is used to calibrate the other EM sensors.

Let P_{OP}⁰(tₖ) and P_{EM}⁰(tₖ_{ᵢ}) be the true position of EM⁰ when measured by the optical and electromagnetic EM⁰ in their respective coordinate frame. The actual measurement of the position of EM⁰ as measured by optical sensor can be represented by

Z_{OP}_{⁰}(tₖ)=P_{OP}_{⁰}(tₖ)+v_{OP}(tₖ) (6)

where v_{OP} is the measurement noise of the optical tracker. v_{OP} is assumed to be Gaussian with zero mean and covariance R_{OP} which is determined using the manufacturer sensor accuracy information. Let Z_{OEM}_{⁰} be the measurement made by the optical sensor of the position of EM⁰ expressed in the EM sensor reference frame:

Z_{OEM}_{⁰}(tₖ)=T_{OPEM}(Z_{OP}_{⁰}(tₖ)) (7)

where T_{OPEM} represents the coordinate transformation matrix from the coordinate frame of the optical sensor to the coordinate frame of the base of the electromagnetic tracker.

In the absence of magnetic disturbances, the measurement provided by EM⁰ is given by

Z_{EM}_{⁰}ⁱ(tₖᵢ)=P_{EM}_{⁰}(tₖᵢ)+v_{EM}(tₖᵢ), tₖ_{ᵢ}=tₖ₁+kᵢT_{EM}, 1≦kᵢ≦n (8)

where v_{EM} models the measurement noise of EM⁰ in the absence of magnetic disturbances. It is assumed to be Gaussian with zero mean and covariance R_{EM} that is determined using the manufacturer accuracy information.

Let

$$V = \frac{{P}_{OP}^{0} ( {t}_{k - 1} + {T}_{OP} ) - {P}_{OP}^{0} ( {t}_{k - 1} )}{{T}_{OP}} \qquad ( 9 )$$

be the velocity of EM⁰ at time tₖ. If the position of EM⁰ at time tₖ_{ᵢ} is P_{EM}_{⁰}(tₖ_{ᵢ}), it will be P_{EM}_{⁰}(tₖ) at time tₖ , where

P_{EM}_{⁰}(tₖ)=P_{EM}_{⁰}(tₖ_{ᵢ})+V(tₖ−tₖ_{ᵢ}) (10)

Note that ideally, one has

P_{OP}⁰(tₖ)=T_{EMOP}(P_{EM}_{⁰}(tₖ)) (11)

In the presence of electromagnetic interferences, the ith measurement of EM⁰ can be modeled as

\tilde{Z}_{EM}_{⁰}ⁱ(tₖᵢ)=P_{EM}_{⁰}(tₖᵢ)+v_{EM}(tₖᵢ)+b, tₖ_{ᵢ}=tₖ₋₁+kᵢT_{EM}, 1≦kᵢ≦n (12)

where b is the bias introduced in the EM sensor measurements due to magnetic distortions. It is assumed that b is constant between two consecutive measurements of the optical sensor. Using Eq. (10), the distorted measurement taken at time tₖ_{ᵢ} when expressed at time tₖ can be written as

$$\begin{matrix}{\tilde{Z}}_{{EM}^{0}}^{i} ( {t}_{k} ) = {P}_{{EM}^{0}} ( {t}_{{k}_{i}} ) + V ( {t}_{k} - {t}_{{k}_{i}} ) + {v}_{EM} ( {t}_{{k}_{i}} ) + b , \\ = {P}_{{EM}^{0}} ( {t}_{k} ) + {v}_{EM} ( {t}_{{k}_{i}} ) + b\end{matrix} \qquad ( 13 )$$

### Defining

δᵢ=\tilde{Z}_{EM}ⁱ(tₖ)−T_{OPEM}(Z_{OP}⁰(tₖ)) (14)

### Using Eqs. (6) and (13), one has

δᵢ=P_{EM}^{OT}(tₖ)+v_{EM}(tₖᵢ)+b−T_{OPEM}(P_{OP}⁰(tₖ)+v_{OP}(tₖ)) (15)

### Using Eq. (11):

δᵢ=b+v_{EM}(tₖᵢ)−T_{OPEM}(v_{OP}(tₖ)) (16)

### Defining

v_{b}=v_{EM}(tₖᵢ)−T_{OPEM}(v_{OP}(tₖ)) (17)

one has

δᵢ=b+v_{b}, i=1, . . . , n (18)

Note that using the previous assumptions on v_{OT} and v_{EM}, v_{b} is zero mean with covariance R_{b}, where

$$\begin{matrix}{R}_{b} = E [ {v}_{b} {v}_{b}^{T} ] = {R}_{EM} + {T}_{OPEM} {R}_{OP} {T}_{OPEM}^{′} & ( 19 ) \\ δ = [ \begin{matrix}{δ}_{1} \\ {δ}_{2} \\ ⋮ \\ {δ}_{n}\end{matrix} ] & ( 20 ) \\ H = [ \begin{matrix}I \\ I \\ ⋮ \\ I\end{matrix} ] & ( 21 ) \\ R = diag ( {R}_{b} ) & ( 22 ) \\ {V}_{b} = [ \begin{matrix}{v}_{b} ( {t}_{1} ) \\ {v}_{b} ( {t}_{2} ) \\ ⋮ \\ {v}_{b} ( {t}_{n} )\end{matrix} ] & ( 23 )\end{matrix}$$

Where I is an identity matrix. Eq. (18) can be rewritten as

δ=Hb+V_{b} (24)

Defining the performance measure J as

J=(δ−Hb)^{T}R⁻¹(δ−Hb) (25)

The estimate of b that minimizes the performance measure J is given by

\hat{b}=(H^{T}R_{b}⁻¹H)⁻¹H^{T}R_{b}⁻¹δ (26)

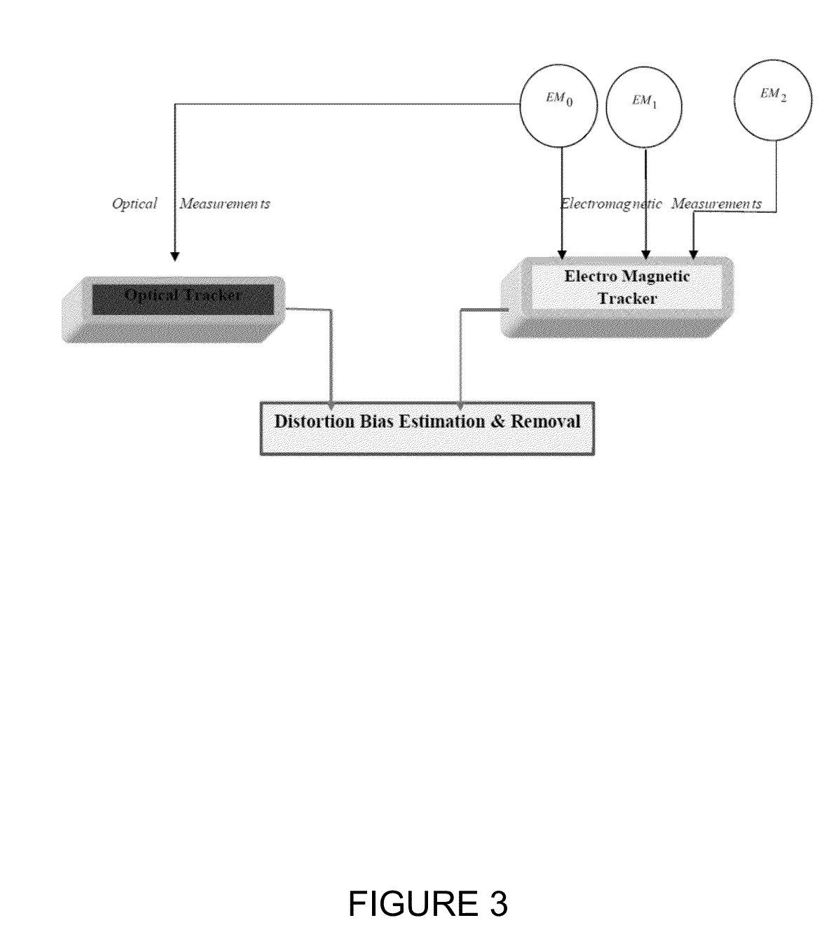

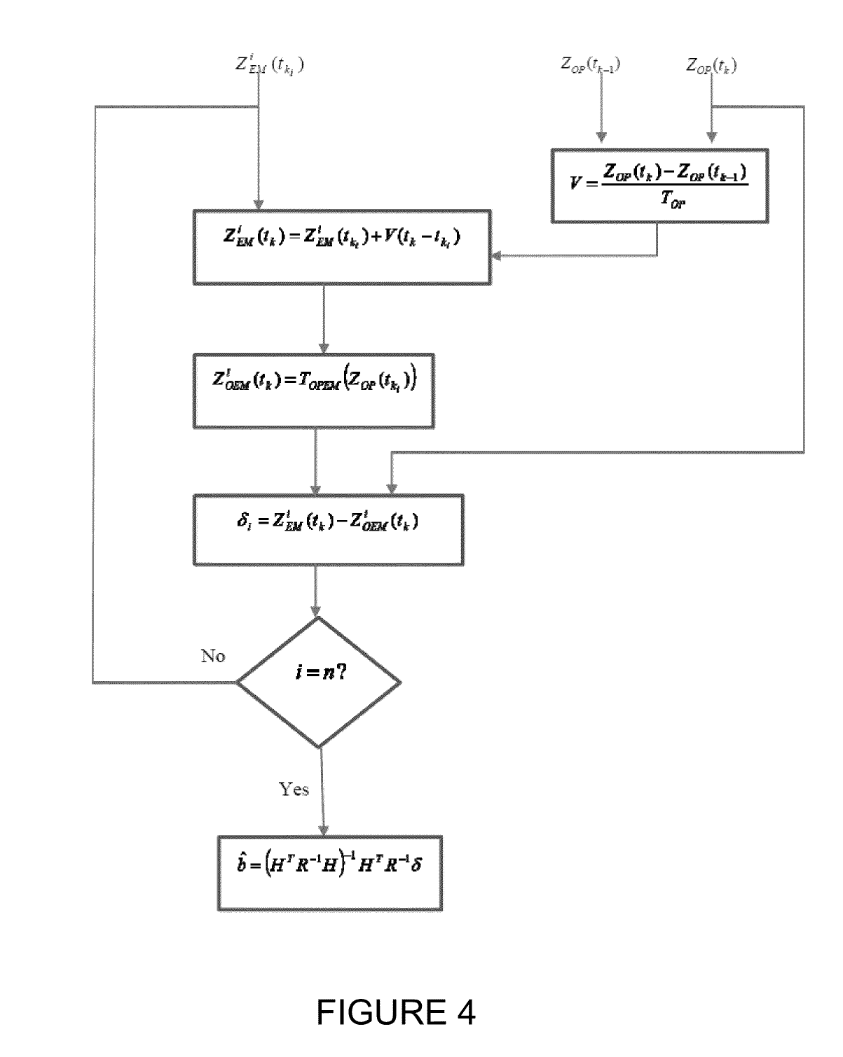

Eq. (26) provides a real time estimate of the magnetic disturbance at a given time and at a given position of the minimally invasive instrument during the surgery. This estimate is used to correct the measurements of the EM sensors before they are used by the tracking system to estimate the position of the tip of the instrument. It is important to notice that the estimate of Eq. (26) can be updated as often as the data rate of the optical sensor. The steps of the online calibration is shown in FIG. 3, with more details provided in FIG. 4.

The online calibration process of the three EM sensors will continue until the deflection of the tip starts to take place. At that time, the dynamic model of the tip of the minimally invasive tool is updated using a maneuvering model and the measurement bias of EM₁ will be used to calibrate future measurements of EM₂.

In order to provide further context for the various aspects of the invention, the following discussion provides a brief, general description of a suitable computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, hand-held devices, microprocessor-based systems, multiprocessor systems, TV set-top boxes and devices, programmable consumer electronics, cell phones, personal digital assistants (PDAs), network PCs, minicomputers, mainframe computers, embedded systems, distributed computing environments, and the like.

Embodiments of the invention may be implemented in the form of computer-executable instructions, such as program code or program modules, being executed by a computer or computing device. Program code or modules may include programs, objections, components, data elements and structures, routines, subroutines, functions and the like. These are used to perform or implement particular tasks or functions. Embodiments of the invention also may be implemented in distributed computing environments. In such environments, tasks are performed by remote processing devices linked via a communications network or other data transmission medium, and data and program code or modules may be located in both local and remote computer storage media including memory storage devices.

In one embodiment, a computer system comprises multiple client devices in communication with at least one server device through or over a network. In various embodiments, the network may be wireless or comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. The client devices each comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CDROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C#, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RAM typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, personal digital assistants, computer notebooks, hand-held devices, cellular phones, mobile phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A method of tracking the tip of a surgical instrument during a surgical procedure, comprising the steps of:
providing a surgical instrument with a plurality of sensors mounted or affixed thereto, wherein some or all of the sensors are asynchronous;
obtaining position data from the sensors that remain in line-of-sight during the surgical procedure;
obtaining position data from the sensors that lose line-of-sight during part or all of the surgical procedure; and
processing, using a computer processor or microprocessor, the position data from the plurality of sensors according to a track fusion algorithm to determine the position of the tip of the surgical instrument.

2. The method of claim 1, further comprising the step of displaying the position of the tip on an image-guided surgical display.

3. The method of claim 1, further comprising the steps of:
receiving data from a real-time imaging modality; and
incorporating the real-time imaging modality data with the tip position data to more accurately determine the position of the tip.

4. The method of claim 3, wherein the real-time imaging modality comprises ultrasound.

5. The method of claim 1, wherein the sensors comprise optical sensors and electromagnetic sensors.

6. The method of claim 5, further wherein the sensors comprise force sensors or sonar sensors.

7. The method of claim 1, wherein the surgical instrument comprises a rigid or fixed section and a flexible section.

8. The method of claim 1, wherein the surgical instrument comprises a minimally invasive surgical instrument.

9. The method of claim 8, wherein the surgical instrument comprises a laparoscopic instrument.

10. A surgical instrument for use with minimally invasive surgical procedures, comprising:
a rigid shaft with a first and second end,
a flexible shaft with a first and second end, the first end of the flexible shaft connected to the second end of the rigid shaft;
a first sensor affixed to the rigid shaft between the first end of the rigid shaft and the approximate middle of the rigid shaft;
a second sensor affixed to the rigid shaft proximate the second end of the rigid shaft;
a third sensor affixed proximate the second end of the flexible shaft.

11. The surgical instrument of claim 10, wherein at least two of the sensors are asynchronous.

12. The surgical instrument of claim 10, wherein the first sensor is an optical sensor or magneto-optical sensor.

13. The surgical instrument of claim 10, wherein the third sensor is an electromagnetic sensor.

14. The surgical instrument of claim 10, further comprising a force sensor affixed proximate to or on the second end of the flexible shaft.
